(19) United States
(12) Patent Application Publication
(10) Pub. No.: US 2019/0189289 A1
(43) Pub. Date: Jun. 20, 2019

(54) System and Method for Using Decision Rules to Identify and Abstract Data from Electronic Health Sources

(71) Applicants: Indra N. Sarkar, Shelburne, VT (US); Elizabeth S. Chen, Shelburne, VT (US); Beth Anderson, Burlington, VT (US); Jeffrey D. Horbar, Charlotte, VT (US); Paul T. Rosenau, Shelburne, VT (US); Matthew B. Storer, Burlington, VT (US)

(72) Inventors: Indra N. Sarkar, Shelburne, VT (US); Elizabeth S. Chen, Shelburne, VT (US); Beth Anderson, Burlington, VT (US); Jeffrey D. Horbar, Charlotte, VT (US); Paul T. Rosenau, Shelburne, VT (US); Matthew B. Storer, Burlington, VT (US)

(21) Appl. No.: 16/284,704

(22) Filed: Feb. 25, 2019

Related U.S. Application Data
(62) Division of application No. 14/855,206, filed on Sep. 15, 2015.

(51) Int. Cl. G16H 50/70; G16H 10/20; G16H 10/60

(52) U.S. Cl. CPC G16H 50/70; G16H 10/60; G16H 10/20

(57) ABSTRACT

The present invention has to do with a method and system for generating condition-specific registries which are essential resources for supporting epidemiological, quality improvement, and clinical trial studies. The identification of potentially eligible patients for a given registry often involves a manual process or use of ad hoc software tools. With the increased availability of electronic health data, such as within Electronic Health Record (EHR) systems, there is potential to develop healthcare standards based approaches for interacting with these data. Arden Syntax, which has traditionally been used to represent medical knowledge for clinical decision support, is one such standard that may be adapted for the purpose of registry eligibility determination.

### CROSS-REFERENCE TO RELATED APPLICATIONS

The present divisional application is related to, claims the earliest available effective filing date(s) from (e.g., claims earliest available priority dates for other than provisional patent applications; claims benefits under 35 USC § 119(e) for provisional patent applications), and incorporates by reference in its entirety all subject matter of the following listed application(s) (the “Related Applications”) to the extent such subject matter is not inconsistent herewith; the present application also claims the earliest available effective filing date(s) from, and also incorporates by reference in its entirety all subject matter of any and all parent, grandparent, great-grandparent, etc. applications of the Related Application(s) to the extent such subject matter is not inconsistent herewith:

U.S. patent application Ser. No. 14/855,206 entitled “A System and Method for Using decision Rules to Identify and Abstract Data from Electronic Health Sources,” naming Indra N. Sarkar as first named inventor, filed 15 Sep. 2015.

### BACKGROUND

### 1. Field of Use

This invention relates generally to systems and methods for developing and using medical logical modules for encoding medical protocols and information, and more particularly concerns a system and method for development and use of medical logic modules to determine registry eligibility based upon disparate computer implemented sources.

### 2. Description of Prior Art (Background)

The Arden Syntax for medical logic modules (MLMs) is a computer programming language for encoding medical knowledge. Each medical logical module typically contains logic or information allowing a user to make one or more medical decisions, and can generate output such as e-mail messages, clinical alerts, interpretations, diagnoses, screening for clinical research, quality assurance functions, and administrative support, for example. With an appropriate computer program, also known as an event monitor, a medical logic module can run automatically, to generate advice as needed. For example, a medical logic module can provide a warning and advice to health care workers when a patient develops new or worsening kidney failure. The Arden Syntax for medical logic modules has been used extensively, for example, at Columbia-Presbyterian Medical Center in New York, and other major medical institutions and universities.

One major functional component of a MLM is to define the context, also termed the evoke slot, in which the medical logical module will be used, such as defining when the medical logical module is pertinent, or whether the medical logical module will be used in conjunction with data storage, another medical logical module, or another application.

Another major functional component of a medical logical module is the logic, or logic slot, such as a set of medical criteria or algorithm, for example, and concluding whether a logical outcome is true or false. The medical logical module can then perform some form of action function, or action slot, to be executed when the logic concludes true, such as to store a message, send e-mail, or return a value, for example. The medical logical module maps the action to a data slot, such as to an institution's local database. For example, medical logical modules can generate a coded or narrative message; a clinical message or alert sent to the provider taking care of a patient; a warning of some concern which is usually flagged in some way; an interpretation or message of advice or information, including diagnosis support; or a screen, which typically results in a message, often sent by e-mail, to a researcher or quality assurance officer informing them of a patient that fits some criteria. Medical logical modules can also trigger each other, and can perform specific actions specific to an institution, such as communicating with another programming application or database.

Arden Syntax, which dates back to 1989, is a Health Level 7 (HL7) maintained standard for representing clinical knowledge such that it may be used to support clinical decision-making. As an HL7 standard, it is regularly updated and supported by a formal working group that oversees the advancement of the standard in accordance to HL7 processes. By maintaining algorithms in self-contained Medical Logic Modules (MLMs), Arden. Syntax provides a means to share decision-making rules independent from technical implementation across institutions or environments. MLMs are organized into major categories (maintenance, library, and knowledge) that store information into “slots.” The maintenance and library categories have slots for metadata associated with the management (maintenance category) and categorization (library category) of a given MLM. The knowledge category contains slots that are used for representing the actual clinical knowledge. For example, the data and logic slots within the knowledge category define the variables that will be used within the MLM (data slot) and the procedural logic that is required for representing the clinical knowledge for the MLM (logic slot).

For an MLM written in Arden Syntax to function, it requires that the source data (usually an electronic health record (EHR)) conform to a usable format. This has historically required custom interfaces for accessing EHR-based data. By contrast, the secondary use of EHR-based data for research purposes commonly involves the extraction of data into research data repositories.

There remain other contexts, however, where the incorporation of such external systems into the health data ecosystem of a clinical enterprise is not feasible or perhaps even permissible. To address this challenge, many EHR vendors provide a “reporting database” that enables one to query data from the EHR using Structured Query Language (SQL).

The increased adoption of EHR systems for the management of patient data has largely been touted for the ability to improve health care outcomes as well as to support research endeavors. An essential first step in the enablement of EHR data to support research is the identification of patient cohorts that match a specified set of criteria. Myriad approaches that leverage healthcare standards have been described for identifying such types of patient cohorts within EHR systems to serve as subjects of prospective clinical trials.

By contrast, there has been limited description of such types of vendor-agnostic approaches that may also be used to populate condition-specific registries from EHR systems. Condition-specific registries provide a population-level view of retrospective data that may originate from clinical encounters. Approaches that may be used for identification of eligible patients for clinical trials could potentially be used for identifying patient data that fit a specified set of criteria for inclusion in a registry.

Many approaches for gathering patient data for registries require laborious and error-prone chart review. Errors can include incorrect entry of data from the chart into the registry, incomplete data entry, and subjective interpretation of objective criteria for determining eligibility.

For example, the Vermont Oxford Network (VON) is a non-profit collaboration that gathers and enables the study of neonatology data from over 900 Neonatal Intensive Care Units (NICUs) that span the globe. The data are gathered for neonates from VON members that meet specified eligibility criteria into de-identified registries that have been used to support a range of activities, including quality improvement projects, clinical trials, and outcomes research. VON members each develop a process for identifying eligible neonates according to specified VON criteria and provide data systematically using common formats or interfaces that are maintained by VON. The source systems increasingly include healthcare enterprises that utilize an EHR for primary clinical data gathering and analysis. Due to the potential range of EHR options, including both vendor and homegrown systems, there is motivation to develop a systematic and uniform process to identify eligible neonates whose data may be contributed into the VON registries.

Therefore, it would be desirable to have a system that leveraged medical knowledge rules, such as those represented in Arden Syntax, to determine the eligibility status of patients who have data in an EHR and furthermore use the system to collect electronic health data for the population of registries, such as the VON Very Low Birth Rate (VLBW) registry.

### BRIEF SUMMARY

The foregoing and other problems are overcome, and other advantages are realized, in accordance with the presently preferred embodiments of these teachings. The subject invention leveraged Arden Syntax for identifying eligible patients from an Epic EHR system deployed at an academic health center. The process for adapting Arden Syntax to be used for identifying eligible records from the Epic EHR is described herein. The performance of the approach is further described based on evaluation relative to a reference standard that included previously identified records for a VON registry.

The invention is directed towards a computer implemented method for determining patient eligibility for inclusion within a condition specific registry. The method includes executing on a processor the steps of predetermining and storing in a filter-rule memory sector a filter rule set for defining at least one condition specific characteristic associated with the condition specific registry. The method also includes developing a patient cohort registry resulting from analyzing a plurality of disparate electronic health care records in accordance with the predetermined filter rule set.

The invention is also directed towards a non-transitory computer readable medium having instructions stored thereon for determining patient eligibility for inclusion within a condition specific registry based upon patient records electronically stored in at least one electronic health reporting database. The instructions, when executed on a processor, perform the steps of encoding, in a first syntax, eligibility criteria, wherein the encoded eligibility criteria is stored in a computer with a memory having a medical logic module; and, interpreting the first syntax with a first parsing routine to generate source code for a first recognizer of the first syntax. The instructions also include steps for parsing relational database management system statements derived from interpreting the first syntax and querying the at least one electronic health database with the parsed statements to determine patient eligibility based upon the query results. The instructions include steps to record query results in computer memory having a registry memory sector.

In accordance with one embodiment of the present invention a non-transitory computer readable medium comprising instructions stored thereon for determining patient eligibility for inclusion within a condition specific registry is provided. The instructions when executed on a processor, perform the steps of encoding, in Arden Syntax, eligibility criteria, wherein the encoded eligibility criteria is stored in a computer with a memory having a medical logic module. The instructions also include, when executed on a processor, the steps of interpreting the Arden Syntax with a first parsing routine to generate source code for a first recognizer of the Arden syntax, wherein interpreting the Arden Syntax with a first parsing routine to generate source code for a first recognizer of the Arden Syntax, further comprises interpreting the Arden Syntax with ANTLR parsing routine to generate source code for a first recognizer of the first syntax. Also included are steps for parsing SQL statements derived from interpreting the Arden syntax, wherein, parsing structured query language (SQL) system statements derived from interpreting the Arden Syntax, further comprises employing an Akiban SQL parser to parse the structured query language (SQL) system statements derived from interpreting the Arden Syntax. Also included are the program steps to query the at least one electronic health database with the parsed statements and determine patient eligibility based upon the query results. The query results are recorded in computer memory having a registry memory sector.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a schematic illustration of a system for using decision rules to identify and abstract data from electronic health sources in accordance with the present invention;

FIG. 2 is a high level schematic illustration of the system shown in FIG. 1 for using decision rules to identify and abstract data from electronic health sources;

FIG. 3 is one example of an eligibility criteria method implemented in the present invention shown in FIG. 1 and FIG. 2;

FIG. 4 is a partial example of a Medical Logic Module implemented in the present invention shown in FIG. 1 and FIG. 2.

### DETAILED DESCRIPTION

The following brief definition of ten is shall apply throughout the application:

The term “comprising” means including but not limited to, and should be interpreted in the manner it is typically used in the patent context;

The phrases “in one embodiment,” “according to one embodiment,” and the like generally mean that the particular feature, structure, or characteristic following the phrase may be included in at least one embodiment of the present invention, and may be included in more than one embodiment of the present invention (importantly, such phrases do not necessarily refer to the same embodiment);

If the specification describes something as “exemplary” or an “example,” it should be understood that refers to a non-exclusive example; and

If the specification states a component or feature “may,” “can,” “could,” “should,” “preferably,” “possibly,” “typically,” “optionally,” “for example,” or “might” (or other such language) be included or have a characteristic, that particular component or feature is not required to be included or to have the characteristic.

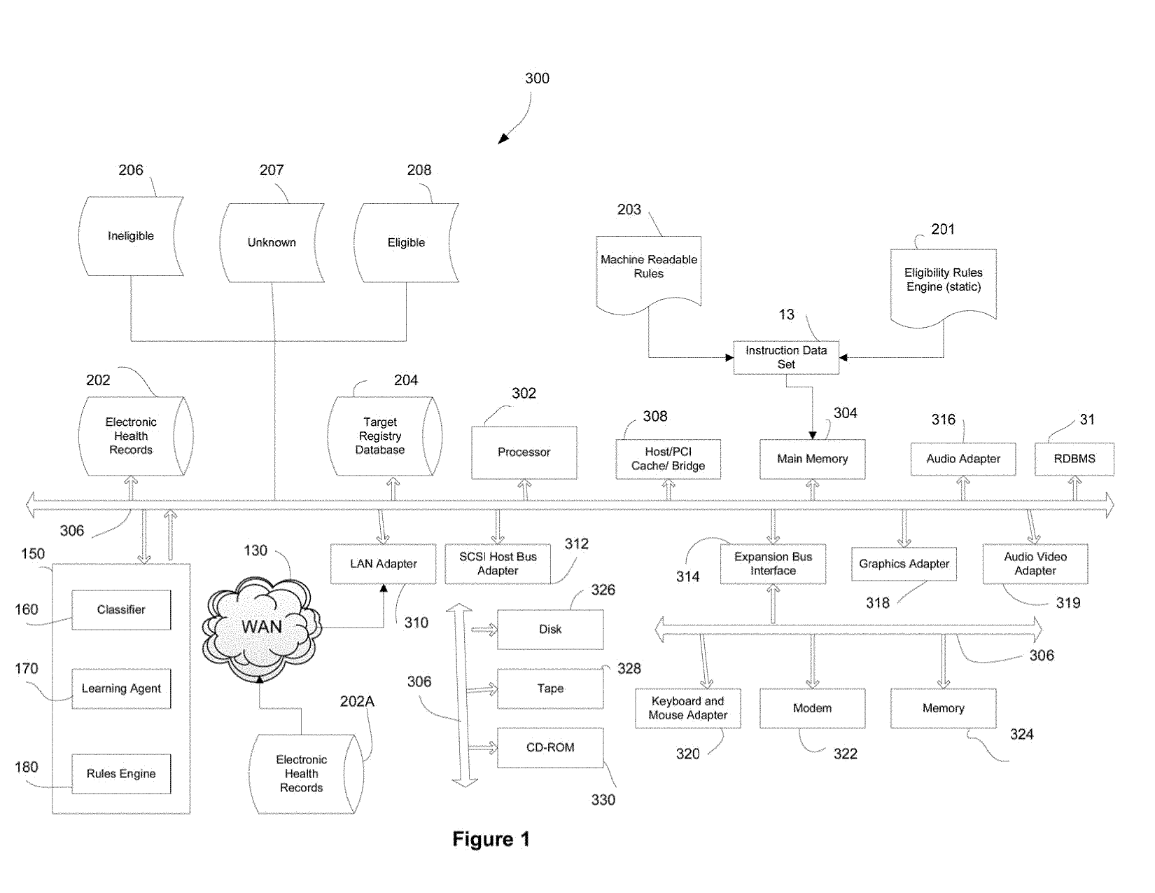

With reference now to FIG. 1, a block diagram illustrating a system 300 for using decision rules to identify and abstract data from electronic health sources is depicted in which the present invention may be implemented. System 300 employs a peripheral component, interconnect (PCI) local bus architecture. Although the depicted example employs a PCI bus, other suitable bus architectures such as Accelerated Graphics Port (AGP) and Industry Standard Architecture (ISA) may be used. Processor 302 and main memory 304 are connected to PCI local bus 306 through PCI bridge 308. PCI bridge 308 also may include an integrated memory controller and cache memory for processor 302. Additional connections to PCI local bus 306 may be made through direct component interconnection or through add-in boards.

In the depicted example, local area network (LAN) adapter 310, SCSI host bus adapter 312, and expansion bus interface 314 are connected to PCI local bus 306 by direct component, connection. It will be understood that LAN adapter 310 may also include an Internet browser and network interface connections for connecting to a wide area network 130, the World Wide Web or Internet for receiving and/or sending instructions and/or data, e.g., patient data. Audio adapter 316, graphics adapter 318, and audio/video adapter 319 are connected to PCI local bus 306 by add-in boards inserted into expansion slots. Expansion bus interface 314 provides a connection for a keyboard and mouse adapter 320, modem 322, and, additional memory 324. Small computer system interface (SCSI) host bus adapter 312 provides a connection for hard disk drive 326, tape drive 328, and CD-ROM drive 330. Typical PCI local bus implementations will support PCI expansion slots or add-in connectors.

An operating system runs on processor 302 and is used to coordinate and provide control of various components within data processing system 31. Data processing system 31 may be configured to process electronic health records 202 and the target registry database 204 as described herein. The operating system may be any suitable commercially available operating system. In addition, an object oriented programming system such as Java may run in conjunction with the operating system and provide calls to the operating system from Java programs or applications executing on data processing system 300. “Java” is a trademark of Sun Microsystems, Inc. Instructions for the operating system, the object-oriented programming system, and applications or programs are located on storage devices, such as hard disk drive 326, and may be loaded into main memory 304 for execution by processor 302.

System 300 may be configured to process decision rules to identify and abstract data from electronic health sources as described herein in real time or store decision rules to identify and abstract data from electronic health sources as described herein in memory 324. Similarly, decision rules to identify and abstract data from electronic health sources, preprocessed or otherwise, may be introduce to system 300 via CD-ROM 300, Tape 328, or Disk 326.

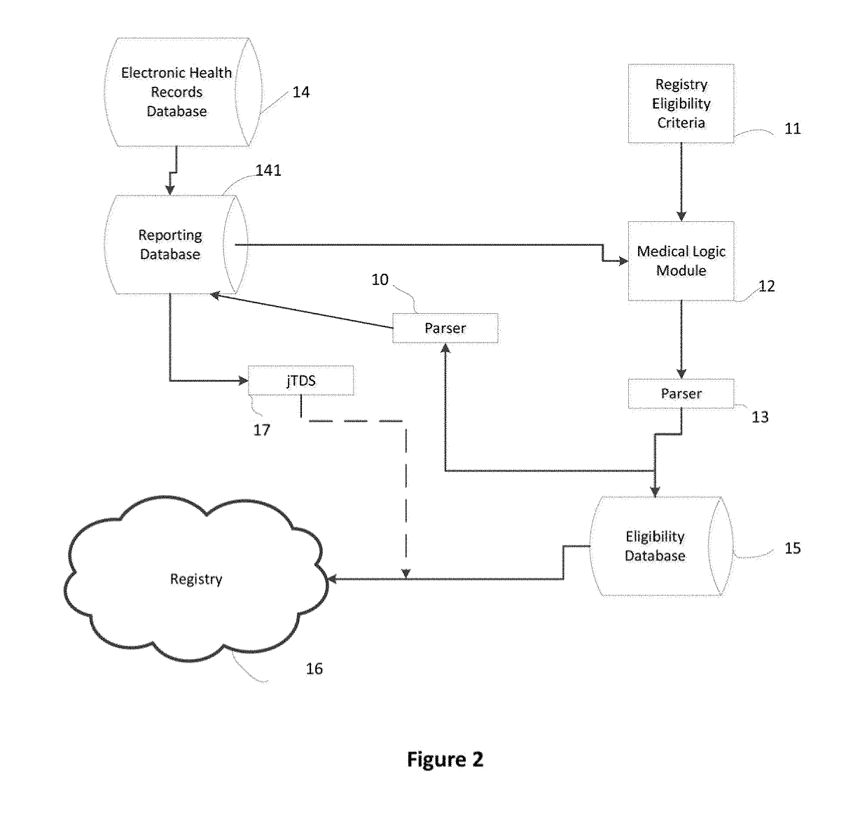

In some embodiments, such an adaptation may be incorporated within system 300. In particular, system 300 may include storage medium 324 with program instructions (see FIG. 2) executable by processor 302 to identify and abstract data from electronic health sources.

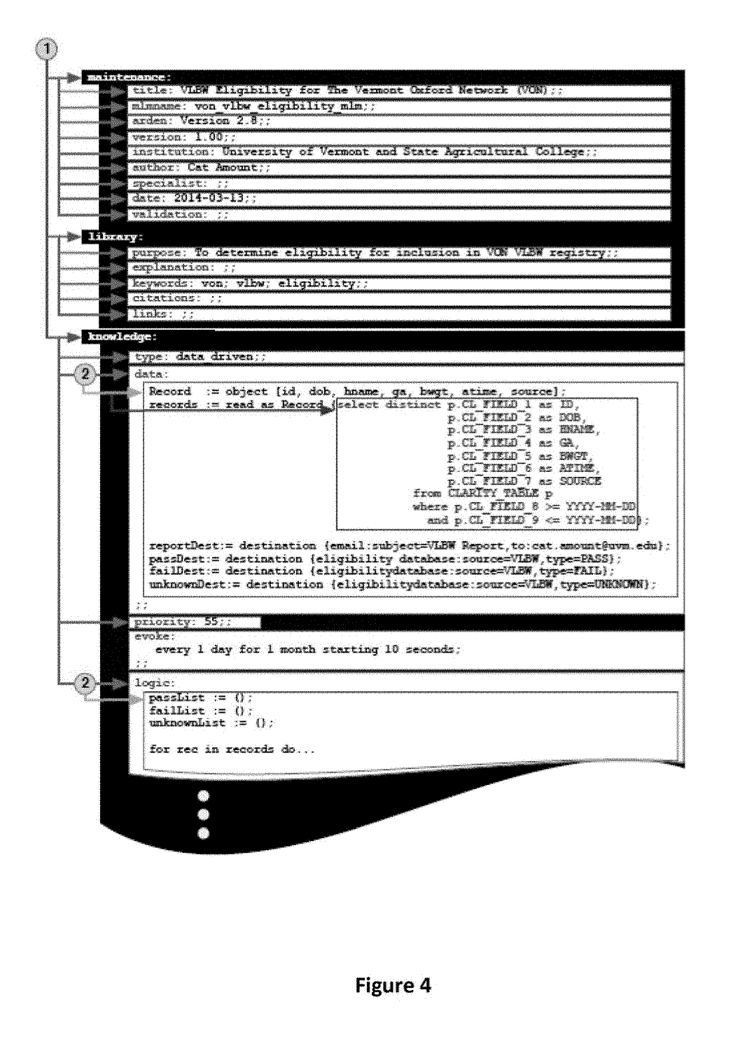

In general, input may be transmitted to system 300 to execute program instructions (see FIG. 4) within storage medium 324. Storage medium 324 may include any device for storing program instructions, such as a read-only memory, a random access memory, a magnetic or optical disk, or a magnetic tape. Program instructions (see FIG. 4) may include any instructions by which to determine and execute the decision rules to identify and abstract data from electronic health sources processes described below. Storage medium 324 also includes a filter rule set sector and a probability algorithm sector.

Those of ordinary skill in the art will appreciate that the hardware in FIG. 1 may vary depending on the implementation. Other internal hardware or peripheral devices, such as flash read-only memory (ROM), equivalent nonvolatile memory, or optical disk drives and the like, may be used in addition to or in place of the hardware depicted in FIG. 1.

Still referring to FIG. 1, Eligibility rules engine 201 contains medical knowledge rules expressed in Arden Syntax, or any suitable rule based language, for determining eligibility. Electronic health records 202 may be locally accessible or EHRs 202A may be available via a wireless or wide area network connection. Machine readable rules 203 are executed by processor 302 to collect and assemble data from EHRs 202, 202A. Machine readable rules may be expressed in any suitable computer language such as, for example, eXtensible Markup Language and Arden Syntax.

Target registry 204 is the registry of data of patients determined to be eligible for the target registry. Processor 302 interprets instruction data set 13, comprising machine readable rules 203 and eligibility rules 201 to categorize patient data as ineligible 206, unknown 207, or eligible 208. Unknown data 207 may be introduced to the target registry via keyboard and Mouse Adapter 320.

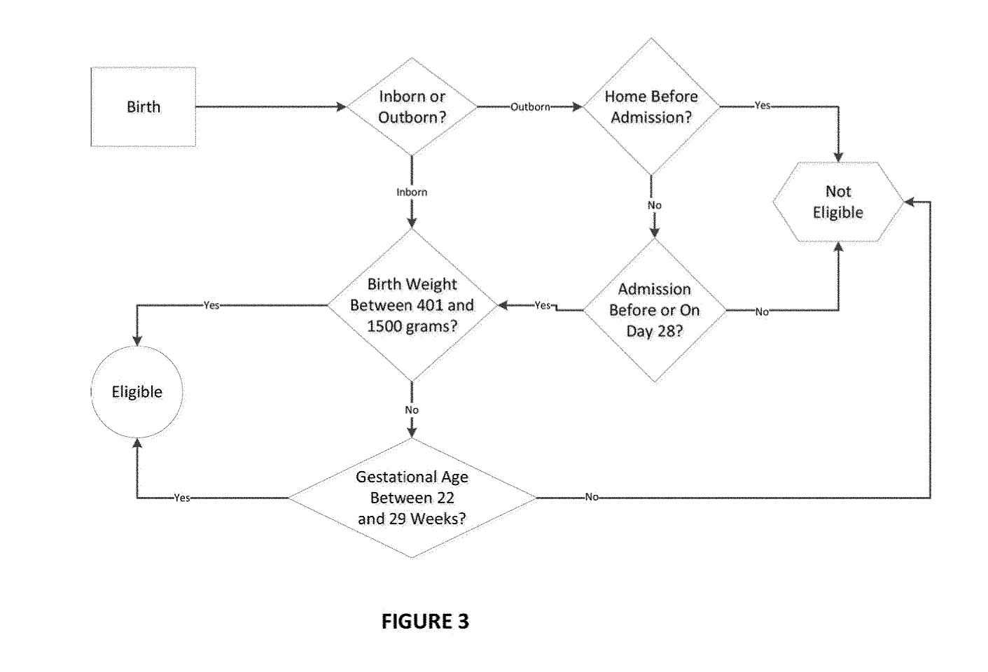

Referring also to FIG. 2 and FIG. 3 there are shown: a high level schematic illustration of the system shown in FIG. 1 for using decision rules to identify and abstract data from electronic health sources; and, one example of an eligibility criteria method implemented in the present invention.

VON VLBW Eligibility Criteria. This particular example focused on the VON Very Low Birth Weight (VLBW) registry, which gathers information on approximately 85% of all VLBW infants born in the US each year. The VON VLBW criteria for eligibility are graphically depicted in FIG. 3.

Briefly, the registry is focused on gathering, data for live-born infants, where status of life is defined as a neonate who breathes or has any evidence for living (e.g., heart beating, umbilical cord pulsation, or definite voluntary muscle movement). Neonates are then categorized into two groups: (1) inborn (where the birth occurs within a particular hospital) and (2) out-born (where the birth occurs outside this hospital but is admitted within 28 days of birth without first having gone home). Additional criteria for inclusion into the VON VLBW registry are defined by birth weight (between 401 and 1500 grams) or gestational age (between 22 and 29 weeks).

Identifying Eligibility Data Fields in the EHR. The framework for the approach developed in this study utilized the Epic Clarity reporting database 141 that contains data extracted from the EHR database 14 (which in this example is a Cache database called Epic Chronicles). It will be understood that any suitable EHR database may be queried. For test purposes, all the data elements required for determining eligibility within the MLM were identified from within the reporting database (i.e., date of birth, hospital name, gestational age, birth weight, admission date/time, and admission source). The chosen data fields were determined based on review of the available data fields in the Epic EHR database 14 and validation of the chosen data fields relative to standard obstetric and neonatology workflows.

A SQL statement retrieved all the data elements in a single query from the Epic Clarity reporting database 141 (equivalent to generating a ‘report’ that included the required data elements).

Representing VON VLBW Eligibility Criteria in Arden Syntax. Within the VLBW MLM data slot (see FIG. 4), a patient record object is created for storing the eligibility data elements needed as well as links to other data needed for retrieving additional information about the patient (e.g., patient identifier). The object is then populated using the aforementioned SQL statement. The eligibility logic as represented by the flow diagram shown in FIG. 3 is encoded into the logic slot of the VLBW MLM 12 using Arden Syntax. Conversions between local system units to those used in the eligibility criteria are also done within the logic slot (e.g., conversion of the birth weight from imperial ounces to metric grams).

To ensure processing of all records retrieved from the EHR, a default categorization of “unknown” is added for patients for whom inborn/out-born status cannot be determined (the first step in the eligibility determination as shown in FIG. 1 after birth); these patients are automatically deemed not eligible.

Developing a System to Process the VLBW MLM. After eligibility criteria are encoded into the MLM 12, a Java-based system 17 interacts with the Epic Clarity reporting database using a jTDS28 Java database connectivity application programming interface.

Eligibility status for records subjected to the VLBW MLM are stored in an eligibility database 15 (either “pass”[eligible], “fail”[not eligible], or “unknown”[not eligible]). For records deemed eligible, an additional Java routine is used to gather electronically available data from Epic Clarity using another set of SQL queries. The resulting records are then transformed into a format that could be transmitted to the VON VLBW registry 16.

The overall approach is shown in FIG. 2 and summarized here. (1) Eligibility criteria 11 are encoded into an Medical Logic Module (MLM) 12; (2) an Another Tool For Language Recognition or ANTLR parser 13 is used to interpret the Arden Syntax (i.e., generates source code for a recognizer of the Arden Syntax); (3) SQL statements are parsed by the Akiban SQL parser 10 and used to send queries to an SQL-92 compliant database 141 and using the jTDS Application Program Interface (API), such as the Epic Clarity reporting database (which is populated from a live EHR database 14, like Epic Chronicles); (4) the returned results of the SQL query are then used by the MLM 12 to determine eligibility; (5) eligible records are recorded in a Eligibility Database 15; (6) the records in the Eligibility Database are enhanced with additional data needed for the registry 16 by additional SQL queries using the jTDS API 17. It will be understood that multiple syntaxes as well as multiple database standards may be used independently or in in conjunction with the process described above. For example, the queried database may be any suitable relational database management system with associated query language and/or flat file databases with associated query formats or languages. Similarly, there may also be multiple EHRs and reporting databases.

The actual processing of the VLBW MLM is a two-phased process, as depicted in FIG. 4. First, an ANTLR parser generator extracts the MLM categories and associated slots. ANTLR uses a context-free grammar (expressed in Extended Backus-Naur Form) to recognize language, generate syntax trees, and process those trees to create machine-interpretable actions. An additional ANTLR parser processes the syntax embedded within specific slots (e.g., the data and logic slots). Version 2.8 of Arden Syntax is the basis for the parsing grammar. It will be readily appreciated that any suitable version of Arden Syntax may be used. Additionally, the Akiban SQL parser interprets embedded SQL-92 compliant SQL statements.

More specifically, and still referring to FIG. 4, MLM Parsing. The invention (1) used an ANTLR parser generator to identify the MLM categories (maintenance, library, and knowledge) and their contained slots; and (2) uses an additional ANTLR parser generator to process the slots and leverage the Akiban SQL Parser to process SQL statements embedded within the data slot. A SQL statement retrieves data from an Epic Clarity reporting database used for eligibility determination (ID=Patient identifier; DOB=patient date of birth; HNAME=hospital name; GA=gestational age; BWGT=birth weight; ATIME=admission time; and SOURCE=admission source) according to the criteria depicted in FIG. 3. It will also be appreciated the ANTLR parser generator may recognize, or identify, a “resources” MLM category.

The MLM parsing results in a parse tree for which a Java routine interprets the retrieved data from the source database (Epic Clarity) and applies the encoded eligibility rules.

System Evaluation. For testing, a reference standard was generated from VON VLBW registry records associated with eligible records. The records contained within the VON VLBW registry for this time period were based on manual chart review and guided by a process workflow.

The VLBW MLM is configured to determine the eligibility of infants born between Jan. 1, 2010 and Dec. 31, 2012. True Positives (TP) are defined as patients who are deemed eligible by the VLBW MLM and in the reference standard; False Positives (FP) are defined as patients who are deemed eligible by the VLBW MLM but not in the reference standard; and False Negatives (FN) are defined as those patients who are in the reference standard but not deemed eligible by the VLBW MLM. The metrics of precision (TP/TP+FP) and recall (TP/TP+FN) are used to assess the overall performance of the approach.

### Test Summary

All of the necessary fields for determining neonate eligibility for inclusion in the VON VLBW registry are identified within the Epic Clarity data schema. The SQL statement that is used to identify the data for all neonates is then embedded within the Arden Syntax within the data slot of the knowledge category of an MLM.

The VON VLBW eligibility criteria are manually encoded into the MLM (within the logic slot of the knowledge category). The instructions for writing the status of a given processed record are defined within the action slot of the knowledge category.

An interpreter interprets the MLMs written in Arden Syntax. For example, utilizing a combination of the ANTLR parsing generator and Akiban SQL parser, a Java-based interpreter interprets MLMs written in Arden Syntax as well as embedded SQL-92 compliant SQL statements. It will be understood that any suitable parser may be used.

The VLBW MLM determines the eligibility of patients born between given dates. In the present test example, the VLBW MLM determined the eligibility of patients born between Jan. 1, 2010 and Dec. 31, 2012. In total, 12,025 neonates who were either born at or transferred to the subject location for that period. The VLBW MLM deemed 192 out of the 12,025 neonates to be eligible for inclusion into the VON VLBW registry. The evaluation was relative to the reference standard, which contained 187 neonates that were manually identified and entered in the VON VLBW registry. The total processing time to determine the eligibility for all newborn records spanning the three-year period was less than five minutes.

### Test Results

Of the eligible patients, 187 were in agreement with the reference standard (TP), five were not found in the reference standard (FP), and no patients in the reference standard were missing from the predicted eligible patient list (FN). The overall precision and recall of the invention was thus determined to be 97.4% and 100.0%, respectively. In alternate embodiments machine learning algorithms may be employed. For example, algorithms used to perform classifications, regressions, clustering, and data density estimations may be used to determine eligibility of the patient data. For example, algorithms such as k-nearest neighbors, naïve Bayes, Support vector machines, decision trees, linear, locally weighted linear, k-means, expectation maximization may be used individually or in combination.

Referring again to FIG. 1. Rule Generator 150 includes Classifier System (CS) 160, Learning Agent 170 and Rules Engine 180 (a probability and rules engine), each of which may include a database. Learning Agent 170 may include any suitable learning algorithm.

Rules Engine 180 contains and supplies the selection rules or in the case of the machine learned classifiers makes calls to Learning Agent 170 to run such classifiers. CS 160 includes logic and resources for eliciting eligibility rules.

It will be appreciated that Rule Generator 150 may work in conjunction with Eligibility Rules Engine 201 or independently. For example each iteration of the Rule Generator using learning or classification algorithms may be used to update or otherwise revise the Eligibility Rules Engine 201.

In the example test evaluation, there were five patients identified by the VLBW MLM but not reported as eligible to the VON VLBW registry (classified in the evaluation as “False Positives”). On closer examination of the data associated with these patients, it was determined that the strict definition of the VON VLBW criteria should have included these individuals. However, human subjectivity resulted in these patients being excluded from the registry. It will be appreciated that an MLM-driven registry eligibility approach as described herein provides a foundation for high-quality and consistent data acquisition.

It will be appreciated that a major potential challenge noted in the prior art is the difficulty in accommodating the heterogeneous nature of EHR implementations across institutions. This issue has been referred to as the “curly-brace problem,” where institution specific coding or mappings required for a given MLM are referenced within a set of curly braces. Amidst some description of systems that are able to directly interact with transactional database systems, the majority of previous implementations of MLM driven decision support systems leveraged local relational databases that contained information from EHR systems.

Other prior art solutions have also included the translation of Arden Syntax MLMs into intermediate formalisms (e.g., Drools). In the approach presented here this challenge was addressed through leveraging EHR (Epic) data through an available SQL-compliant database (Epic Clarity) using SQL-92 compliant statements. It, will be appreciated that the Arden. Syntax, as described herein, is for the population of a registry. Further, the requisite data elements are derived from information that is commonly captured within a clinical chart and are available within a reporting interface that is updated nightly. This is in contrast to more typical prior art scenarios where MLMs are used for more real-time clinical decision support, and would thus need to interface directly with the live EHR system.

Thus, the results demonstrate the application of Arden. Syntax for retrospective patient data eligibility determination and the potential utility for other contexts, such as retrospective determination of patient cohorts that may be eligible for clinical trials or who may need to be systematically alerted about a potential adverse event.

It will also be appreciated that the invention is also able to gather and/or interpret additional data beyond the information needed for determining eligibility using SQL statements and export the full set of gathered data into a number of consumable formats (e.g., as eXtensible Markup Language or a Comma-Separated Value file).

It should be understood that the foregoing description is only illustrative of the invention. Thus, various alternatives and modifications can be devised by those skilled in the art without departing from the invention. For example, Arden Syntax as described herein may be used to represent eligibility criteria and thus provide a mechanism to leverage electronically available health data (e.g., as encapsulated within an EHR) for supporting the population of patient cohort databases such as condition-specific registries. Accordingly, the present invention is intended to embrace all such alternatives, modifications and variances that fall within the scope of the appended claims.

1-6. (canceled)

7. A non-transitory computer readable medium for determining data, eligibility for inclusion within a condition specific registry based upon data records electronically stored in at least one electronic health reporting database, comprising instructions stored thereon, that when executed on a processor, perform the steps of:
encoding, in a first syntax, data eligibility criteria, wherein the encoded data eligibility criteria is stored in a computer with a memory having a medical logic module;
interpreting the first syntax with a first parsing routine to generate source code for a first recognizer of the first syntax;
parsing relational database management system statements derived from interpreting the first syntax;
querying the at least one electronic health database with the parsed statements;
determining patient eligibility data based upon the query results; and
recording query results in computer memory having a registry memory sector.

8. The non-transitory computer readable medium as in claim 7 wherein encoding, in the first syntax, data eligibility criteria, wherein the encoded data eligibility criteria is stored in the computer with the memory having a medical logic module, further comprises:
encoding, in Arden syntax, data eligibility criteria, wherein the encoded data eligibility criteria is stored in a computer with a memory having a medical logic module.

9. The non-transitory computer readable medium as in claim 7 wherein interpreting the first syntax with a first parsing routine to generate source code for a first recognizer of the first syntax, further comprises:
interpreting the first syntax with ANTLR parsing routine to generate source code for a first recognizer of the first syntax;

10. The non-transitory computer readable medium as in claim 7 wherein parsing relational database management system statements derived from interpreting the first syntax, further comprises:
parsing structured query language (SQL) system statements derived from interpreting the first syntax.

11. The non-transitory computer readable medium as in claim 10 wherein parsing structured query language (SQL) system statements derived from interpreting the first syntax, further comprises:
employing an Akiban SQL parser to parse the structured query language (SQL) system statements derived from interpreting the first syntax.

12. The non-transitory computer readable medium as in claim 7 further comprising:
encoding, in a second syntax, data eligibility criteria, wherein the second encoded data eligibility criteria is stored in the computer with the memory having a medical logic module;
interpreting, the second syntax with a second parsing routine to generate source code for a second recognizer of the second syntax;
parsing query statements derived from interpreting the second syntax;
querying the at least one electronic health database with the parsed statements;
determining patient data eligibility based upon the query results; and
recording patient data eligibility and associated query results in the computer memory having the eligibility database memory sector.

13. The non-transitory computer readable medium as in claim 7 further comprising:
enhancing query results with a second set of query statements operating, on the at least one electronic health database; and
recording the enhanced query results in the computer memory having the registry memory sector.

14. The non-transitory computer readable medium as in claim 7 wherein the condition specific registry is very low birth rate neonates.

15. A non-transitory computer readable medium for determining data eligibility for inclusion within a condition specific registry, comprising instructions stored thereon, that when executed on a processor, perform the steps of:
encoding, in Arden Syntax, data eligibility criteria, wherein the encoded data eligibility criteria is stored in a computer with a memory having, a medical logic module;
interpreting the Arden Syntax with a first parsing routine to generate source code for a first recognizer of the Arden syntax, wherein interpreting the Arden Syntax with a first parsing routine to generate source code for a first recognizer of the Arden Syntax, further comprises:
interpreting the Arden Syntax with ANTLR parsing routine to generate source code for a first recognizer of the first syntax;
parsing SQL statements derived from interpreting the Arden syntax, wherein parsing structured query language (SQL) system statements derived from interpreting the Arden Syntax, further comprises:
employing an Akiban SQL parser to parse the structured query language (SQL) system statements derived from interpreting the Arden Syntax;
querying the at least one electronic health database with the parsed statements;
determining data eligibility based upon the query results; and
recording query results in computer memory having a registry memory sector.

16. The non-transitory computer readable medium as in claim 15 further comprising:
encoding, in a second syntax, data eligibility criteria, wherein the encoded data eligibility criteria is stored in the computer with the memory having the medical logic module;
interpreting the second syntax with a second parsing routine to generate source code for a second recognizer of the second syntax;
parsing query statements derived from interpreting the second syntax;
querying the at least one electronic health database with the parsed statements;
determining data eligibility based upon the query results; and
recording data eligibility in computer memory having the eligibility database memory sector.

17. The non-transitory computer readable medium as in claim 15 further comprising:
enhancing query results with a second set of query statements operating on the at least one electronic health database; and
recording enhanced query results in computer memory having the registry memory sector.
